(19) United States
(12) Patent Application Publication
(10) Pub. No.: US 2015/0037250 A1
(43) Pub. Date: Feb. 5, 2015

(54) CONTRAST AGENT

(71) Applicants: Kabushiki Kaisha Toshiba, Minato-ku (JP); Toshiba Medical Systems Corporation, Otawara-shi (JP)

(72) Inventor: Hitoshi YAMAGATA, Otawara (JP)

(73) Assignees: Kabushiki Kaisha Toshiba, Minato-ku (JP); Toshiba Medical Systems Corporation, Otawara-shi (JP)

(21) Appl. No.: 14/447,841

(22) Filed: Jul. 31, 2014

(30) Foreign Application Priority Data
Jul. 31, 2013 (JP) 2013-159854
Jul. 31, 2013 (JP) 2013-159855

(51) Int. Cl. A61K 51/12; G01N 33/50; A61K 49/22; A61K 49/00; A61K 49/04; A61K 49/18

(52) U.S. Cl. CPC A61K 51/1241; A61K 49/04; A61K 49/0438; A61K 49/1818; A61K 49/227; A61K 49/0002; G01N 33/5091
USPC 424/1.29; 424/9.42; 424/9.4; 424/9.3; 424/9.36; 424/9.51; 435/29

(57) ABSTRACT

According to one embodiment, a contrast agent includes a blood vessel contrast enhancement particles configured to enhance contrast of a blood vessel of an object and a diseased tissue contrast enhancement particles configured to enhance contrast of a diseased tissue of the object. The blood vessel contrast enhancement particles have a first particle size larger than a gap of vascular endothelial cells under an EPR effect. The diseased tissue contrast enhancement particles have a second particle size smaller than the gap.

### CROSS-REFERENCE TO RELATED APPLICATIONS

This application is based upon and claims the benefit of priority from the prior Japanese Patent Application No. 2013-159854, filed Jul. 31, 2013 and No. 2013-159855, filed Jul. 31, 2013 the entire contents of all of which are incorporated herein by reference.

### FIELD

Embodiments described herein relate generally to a contrast agent.

### BACKGROUND

In the field of medical imaging such as PET (positron emission tomography), MRI (magnetic resonance imaging), and ultrasonic imaging, many clinical researches have been made on molecular imaging using nanoparticle contrast agents (see, for example, M. F. Kircher and J. K. Willmann, “Molecular Body Imaging: MR Imaging, CT, and US. Part I. Principles”, Radiology, Vol. 263, pp. 633-643, 2012). In addition, clinical researches have been made on molecular imaging using nanoparticles and the like including gold as a heavy metal in PCCT (photon counting CT) called next-generation CT (computed tomography) (see, for example, M. Shilo, et al., “Nanoparticles as computed tomography contrast agents: current status and future perspectives”, Nanomedicine, Vol. 7, pp. 257-269, 2012).

The EPR (enhanced permeability and retention) effect occurs in neighboring blood vessels and new nutrient vessels for cancer cells which have progressed to a certain degree. The EPR effect is a phenomenon in which the enhancement of vascular permeability due to the expansion of the gaps between vascular endothelial cells occurs together with the enhancement of the retention of vascular permeability substances due to the undevelopment of a lymphoid system. It is known that vascular endothelial cell gaps are about 5 nm to 50 nm in a normal state, whereas vascular endothelial cell gaps are about 150 nm or more under the EPR effect. In molecular imaging using nanoparticles, imaging is basically performed by using nanoparticles having a single particle size even with slight variations. For this reason, if the particle size is smaller than a vascular endothelial cell gap, it is difficult to image the blood vessel itself, even though it is possible to image the stromal system of a cancer tissue. In contrast to this, if the particle size is larger than a vascular endothelial cell gap, it is difficult to image the the stromal system of a cancer tissue, even though it is possible to image the blood vessel itself.

### BRIEF DESCRIPTION OF THE DRAWING

FIG. 1 is a view for explaining the EPR effect in a cancer tissue, schematically showing an anatomical structure around the cancer tissue;

FIG. 2 is a view schematically showing the anatomical structure of a vascular system including a region where no EPR effect has occurred in FIG. 1;

FIG. 3 is a view schematically showing the anatomical structure of a vascular system including a region where the EPR effect has occurred in FIG. 1;

FIG. 4 is a view schematically showing an example of a blood vessel contrast enhancement particle and a cancer contrast enhancement particle according to an embodiment;

FIG. 5 is a view schematically showing the behaviors of a blood vessel contrast enhancement particle and a cancer contrast enhancement particle at the time of the occurrence of the EPR effect according to this embodiment;

FIG. 6 is a view schematically showing an example of a blood vessel contrast enhancement particle according to this embodiment;

FIG. 7 is a view schematically showing an example of a cancer contrast enhancement particle according to this embodiment;

FIG. 8 is a view schematically showing the behaviors of a blood vessel contrast enhancement particle and a cancer contrast enhancement particle which are variously modified at the time of the occurrence of the EPR effect according to this embodiment;

FIG. 9 is a view for exemplarily showing a contrast enhancement particle according to this embodiment, schematically showing a contrast enhancement particle when a carrier is a liposome;

FIG. 10 is a view for exemplarily showing a contrast enhancement particle according to this embodiment, schematically showing a contrast enhancement particle when a carrier is a polymer micelle;

FIG. 11 is a view for exemplarily showing a contrast enhancement particle according to this embodiment, schematically showing a contrast enhancement particle when a carrier is a dendrimer;

FIG. 12 is a view showing the comparisons between the particle size and crushing frequency of a blood vessel contrast enhancement particle and those of a cancer contrast enhancement particle contained in an ultrasonic contrast agent according to a modification of this embodiment;

FIG. 13 is a view showing the comparison between a crushing frequency ft of a blood vessel contrast enhancement particle and that of a cancer contrast enhancement particle in a case in which the reperfusion of the cancer contrast enhancement particle in FIG. 12 into a cancer tissue is an observation target;

FIG. 14 is a view schematically showing the behaviors of blood vessel contrast enhancement particles and cancer contrast enhancement particles in the stage of accumulation of cancer contrast enhancement particles in a cancer tissue according to a modification of this embodiment;

FIG. 15 is a view schematically showing the behaviors of blood vessel contrast enhancement particles and cancer contrast enhancement particles in the stage of transmission of a crushing frequency according to a modification of this embodiment; and

FIG. 16 is a view schematically showing the behaviors of blood vessel contrast enhancement particles and cancer contrast enhancement particles in the stage of accumulation of cancer contrast enhancement particles in a cancer tissue according to a modification of this embodiment.

### DETAILED DESCRIPTION

In general, according to one embodiment, a contrast agent includes blood vessel contrast enhancement particles for enhancing the contrast of a blood vessel of an object and diseased tissue contrast enhancement particles for enhancing the contrast of a diseased tissue of the object. The blood vessel contrast enhancement particles have the first particle size larger than the gap between vascular endothelial cells under the EPR effect. The diseased tissue contrast enhancement particles have the second particle size smaller than the gap.

A contrast agent according to this embodiment will be described below with reference to the accompanying drawing.

The contrast agent according to this embodiment is related to a contrast agent used in the medical imaging field. The contrast agent according to the embodiment has the property capable of individually targeting the vascular system and stromal system of a diseased tissue. A diseased tissue whose contrast is to be enhanced by the contrast agent according to the embodiment may be any type of diseased tissue in which the EPR effect occurs in a neighboring blood vessel and a new nutrient vessel with the progress of the lesion. In addition, a diseased tissue may be an inflammatory reaction tissue like the tissue under the EPR effect, in which immune cells like granulocytes discharge cytokines to neighboring vascular endothelial cells to reduce their volumes in the early development of inflammation, and as a result, the gaps between vascular endothelial cells increase, leading to the accentuation of vascular permeability. For the sake of a concrete description to be made below, assume that a diseased tissue is a cancer tissue.

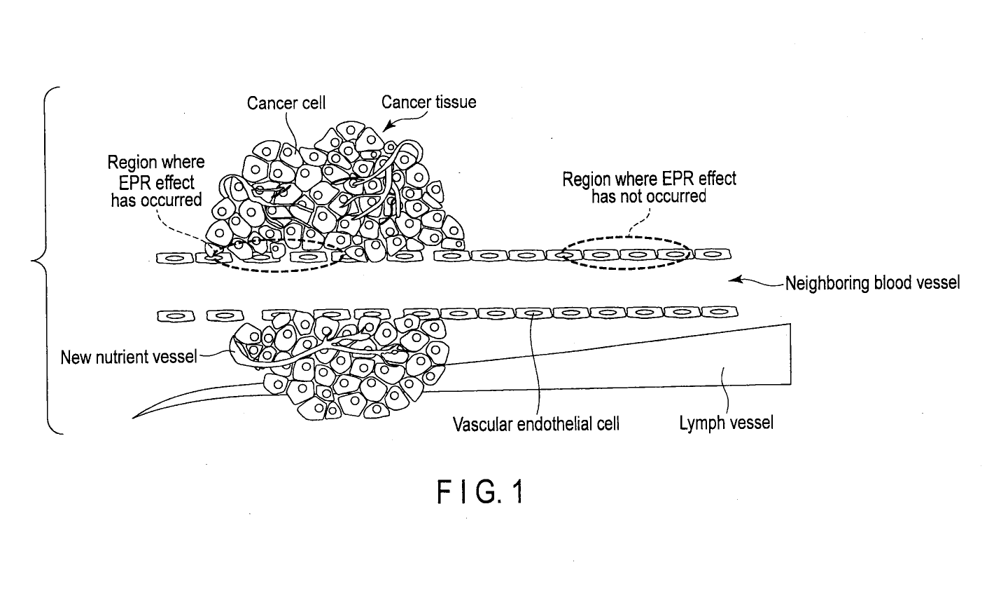

The EPR effect in a cancer tissue will be described below with reference to FIG. 1. FIG. 1 is a view schematically showing an anatomical structure around a cancer tissue. As shown in FIG. 1, the cancer tissue includes a plurality of cancer cells and receives nutrients from a neighboring blood vessel and a new nutrient vessel. The gaps between a plurality of cancer cells are filled with an interstitial fluid (not shown). A blood vessel wall includes a plurality of vascular endothelial cells. Gaps are provided between vascular endothelial cells, and nutrient components and the like flowing in the blood vessel pass through the gaps and are supplied to cancer cells and the like through interstitial fluid. The gaps between vascular endothelial cells will be referred to as vascular endothelial cell gaps hereinafter.

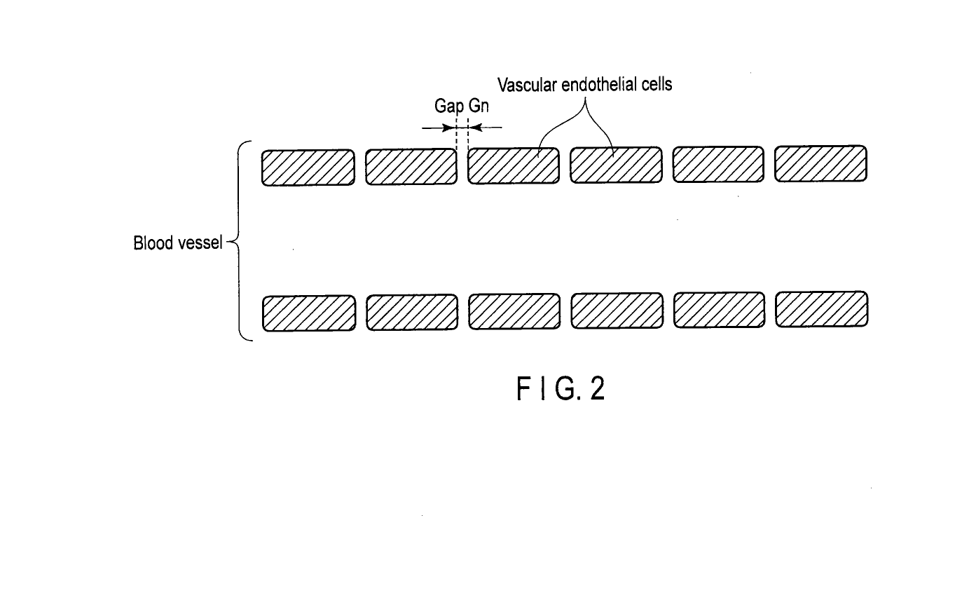

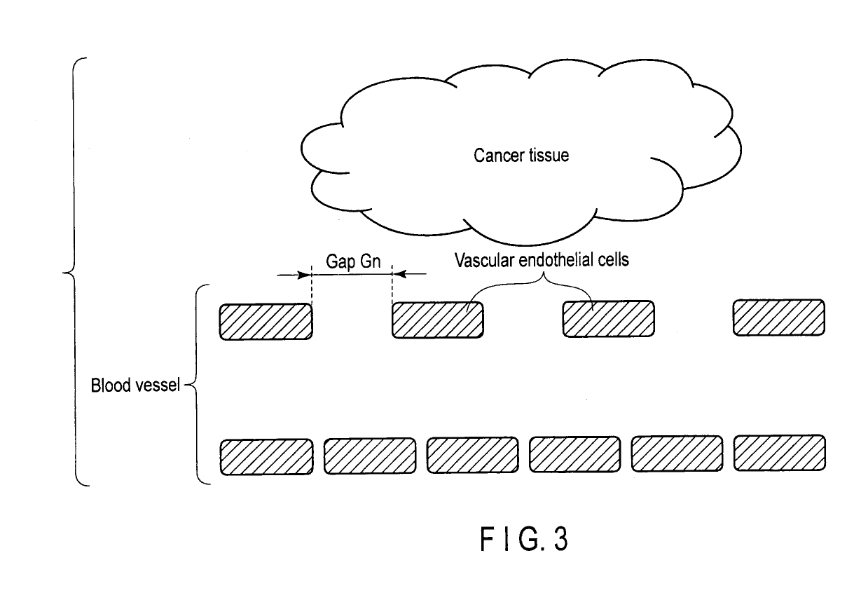

With the progress of cancer, the EPR effect occurs in a neighboring blood vessel or new nutrient vessel for cancer cells. FIG. 2 is a view schematically showing the anatomical structure of a vascular system of a region where no EPR effect has occurred. FIG. 3 is a view schematically showing the anatomical structure of a vascular system of a region where the EPR effect has occurred. As shown in FIGS. 2 and 3, with the progress of cancer, the vascular endothelial cells contract, and the vascular endothelial cell gaps expand. As shown in FIG. 2, a vascular endothelial cell gap Gn in a normal state is typically about 5 nm to 50 nm. As shown in FIG. 3, however, a vascular endothelial cell gap Ga of a blood vessel in which the EPR effect has occurred is larger than the vascular endothelial cell gap Gn in a normal state and expands to about 150 nm or more.

As a conventional contrast agent, contrast enhancement particles having a single particle size are basically used. If contrast enhancement particles having a particle size smaller than a vascular endothelial cell gap are used to image the stromal system of a cancer tissue, since the contrast enhancement particles pass through the vascular endothelial cell gaps and reach the cancer tissue through the interstitial fluid, it is possible to clearly enhance the contrast of the stromal system of the cancer tissue. However, the contrast enhancement effect provided for the blood vessel by the contrast enhancement particles is weakened. In contrast to this, if contrast enhancement particles having a particle size larger than a vascular endothelial cell gap are used to image the vascular system, it is difficult for the contrast enhancement particles to pass through the vascular endothelial cell gaps. This weakens the contrast enhancement effect for the stromal system of the cancer tissue, although it is possible to clearly enhance the contrast of the blood vessel. Note that contrast enhancement particles are nanoparticles having a contrast enhancement effect in the imaging principle of a modality (imaging mechanism) used for imaging with the contrast agent. The contrast enhancement effect indicates the property capable of producing a clear contrast between the contrast agent portion and the non-contrast-agent portion on the medical image acquired by a modality when the contrast agent is imaged by the modality according to a given imaging principle.

The contrast agent according this embodiment has the property capable of individually targeting the vascular system and stromal system of a cancer tissue. That is, the contrast agent according to the embodiment contains a plurality of contrast enhancement particles for enhancing the contrast of a blood vessel and a plurality of contrast enhancement particles for enhancing the contrast of a cancer tissue. In the following description, contrast enhancement particles for enhancing the contrast of a blood vessel will be referred to as blood vessel contrast enhancement particles, and contrast enhancement particles for enhancing the contrast of a cancer tissue will be referred to as cancer contrast enhancement particles.

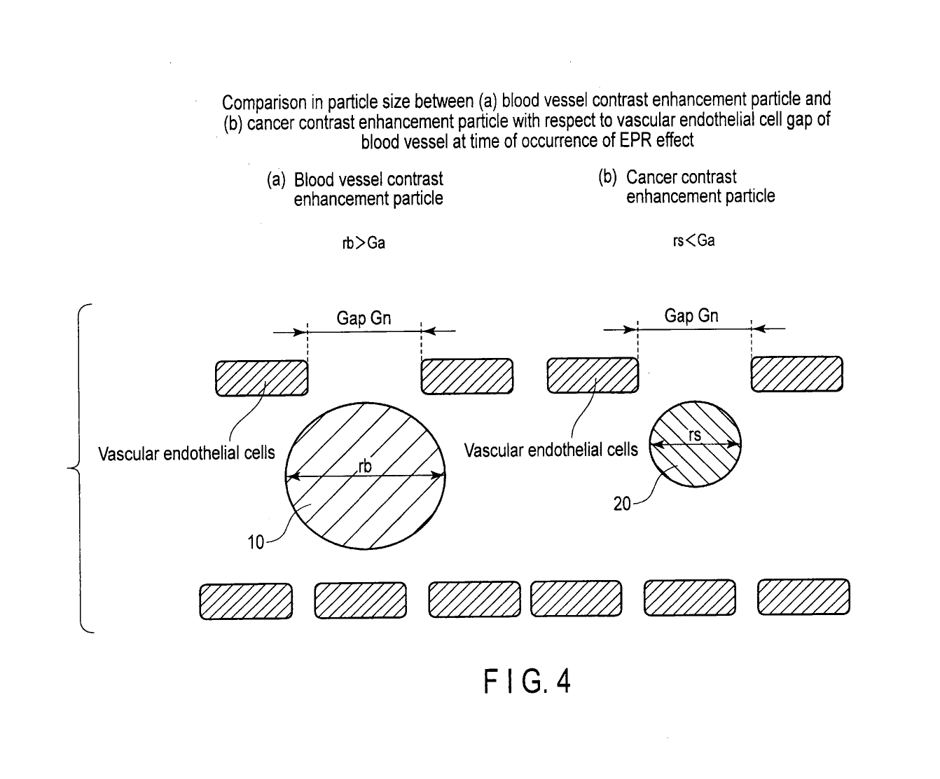

FIG. 4 is a view schematically showing an example of a blood vessel contrast enhancement particle 10 and a cancer contrast enhancement particle 20. As shown in FIG. 4, the blood vessel contrast enhancement particle 10 is formed to have a particle size larger than the vascular endothelial cell gap Ga of an imaging target blood vessel in which the EPR effect has occurred. The cancer contrast enhancement particle 20 is formed to have a particle size smaller than the gap Ga of the imaging target blood vessel in which the EPR effect has occurred.

The particle sizes of the blood vessel contrast enhancement particle 10 and cancer contrast enhancement particle 20 will be described in more detail. The blood vessel contrast enhancement particle 10 and the cancer contrast enhancement particle 20 are almost simultaneously injected into a vein of an object. The vascular endothelial cell gaps of a blood vessel in which the EPR effect has occurred are typically 150 nm or more. Therefore, the particle size of the blood vessel contrast enhancement particle 10 needs to be at least 150 nm or more to prevent the particle from passing through the vascular endothelial cell gap of the blood vessel at the time of the occurrence of the EPR effect. The particle size of the blood vessel contrast enhancement particle 10 is preferably 200 nm or more, more preferably, 300 nm or more to reliably prevent the particle from passing through the vascular endothelial cell gap at the time of the occurrence of the EPR effect. Setting the particle size of the blood vessel contrast enhancement particle 10 in this manner can make the blood vessel contrast enhancement particle 10 retain in the blood vessel without passing through the vascular endothelial cell gap even at the time of the occurrence of the EPR effect.

On the other hand, the cancer contrast enhancement particle 20 is formed to have a particle size equal to or less than 150 nm at most so as to allow the particle to pass through the vascular endothelial cell gap at the time of the occurrence of the EPR effect. Macrophages as phagocytes exist in an RES (Reticulo-Endothelial System) of the liver, spleen, or the like. Macrophages are cells which phagocyte foreign substances. In general, contrast enhancement particles circulate in the body. In order to prevent the cancer contrast enhancement particle 20 passing through the vascular endothelial cell gap from being phagocyted by macrophages, the cancer contrast enhancement particle 20 is preferably formed to have a particle size equal to or less than 100 nm. Setting the particle size of the cancer contrast enhancement particle 20 in this manner can more reliably accumulate the cancer contrast enhancement particle 20 in the cancer tissue. Note that when making the cancer contrast enhancement particle 20 pass through the vascular endothelial cell gap only at the time of the occurrence of the EPR effect, the cancer contrast enhancement particle 20 is preferably formed to have a particle size equal to or more than 50 nm.

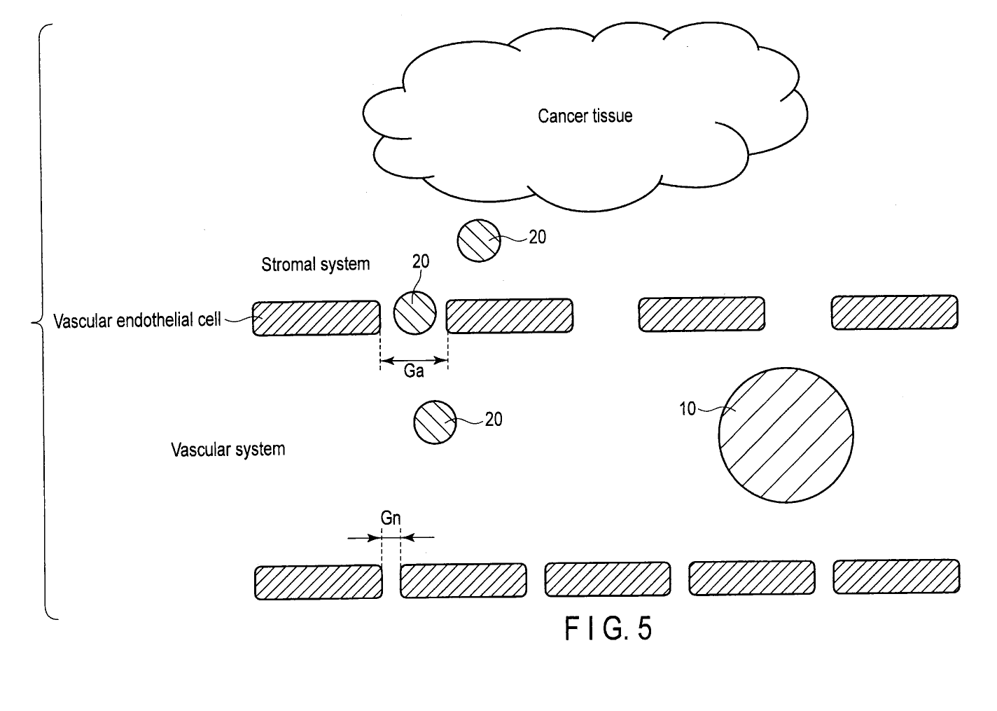

FIG. 5 is a view schematically showing the behaviors of the blood vessel contrast enhancement particle 10 and cancer contrast enhancement particle 20 at the time of the occurrence of the EPR effect. As shown in FIG. 5, the blood vessel contrast enhancement particle 10 and the cancer contrast enhancement particle 20 are individually set in accordance with the standard value of the vascular endothelial cell gap Ga at the time of the occurrence of the EPR effect such that the blood vessel contrast enhancement particle 10 is retained in the blood vessel at the time of the occurrence of the EPR effect, and the cancer contrast enhancement particle 20 is accumulated in the cancer tissue at the time of the occurrence of the EPR effect. Vascular endothelial cell gaps vary depending on the anatomical region in which a blood vessel exists as well as the presence/absence of the EPR effect and the degree of progress of a lesion. Therefore, the particle sizes of the blood vessel contrast enhancement particle 10 and cancer contrast enhancement particle 20 may be decided in accordance with the standard value of the gap Ga for each anatomical region. This makes it possible to individually target the vascular system and stromal system of a cancer tissue independently of an imaging target region. Note that the standard value of the gap Ga for each anatomical region may be determined experimentally and empirically.

Various modifications are preferably provided for the blood vessel contrast enhancement particle 10 and the cancer contrast enhancement particle 20. The modifications provided for the blood vessel contrast enhancement particle 10 and the cancer contrast enhancement particle 20 will be described below.

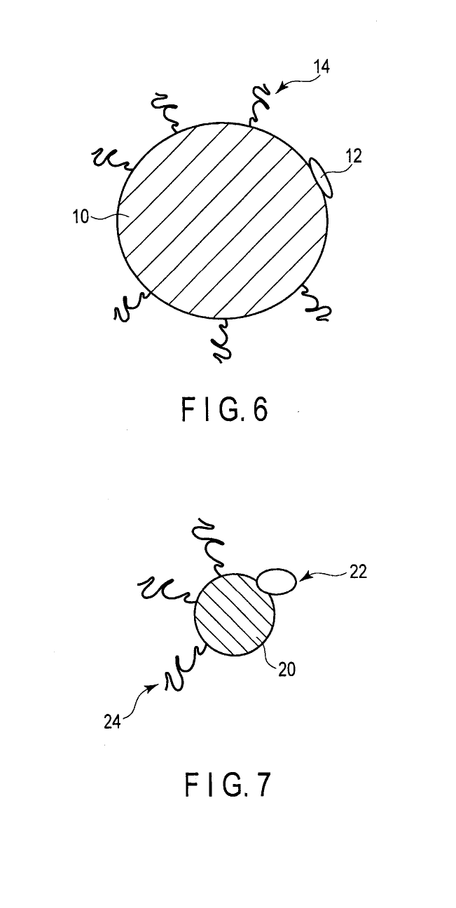

FIG. 6 is a view schematically showing an example of the blood vessel contrast enhancement particle 10. As shown in FIG. 6, the surface of the blood vessel contrast enhancement particle 10 is chemically modified with a functional group 12 which can be bonded to albumin existing in blood. The functional group 12 which can be bonded to albumin existing in blood includes, for example, carbonyl, ether, amide, and amine. If the blood vessel contrast enhancement particle 10 is not bonded to albumin existing in blood of the object, the blood vessel contrast enhancement particle 10 is discharged from the blood vessel by the kidney. For this reason, the blood vessel contrast enhancement particle 10 cannot be retained in the blood vessel for a long period of time. If the blood vessel contrast enhancement particle 10 is bonded to albumin through the functional group 12, the blood vessel contrast enhancement particle 10 is suppressed from being discharged from the blood vessel by the kidney. This allows the blood vessel contrast enhancement particle 10 to be retained in the blood vessel for a long period of time. That is, it is possible to use the blood vessel contrast enhancement particle 10 as a blood pool agent.

FIG. 7 is a view schematically showing an example of the cancer contrast enhancement particle 20. As shown in FIG. 7, a specific ligand 22 is bonded to the surface of the cancer contrast enhancement particle 20. The ligand 22 has the property of specifically adsorbing a specific protein (receptor) existing on the surface of a cancer cell or in the cancer cell. The type of ligand 22 is changed in accordance with the characteristics of a cancer cell of an organ as a contrast enhancement target. As the ligand 22, for example, an EGF (epidermal growth factor) or a VEGF (vascular endothelial growth factor) is used. Providing the ligand 22 in accordance with the characteristics of a cancer cell of an organ as a contrast enhancement target in this manner can specifically accumulate the cancer contrast enhancement particle 20 in the cancer tissue.

In addition, as shown in FIGS. 6 and 7, PEGs (polyethylene glycols) 14 and 24 are preferably formed on the surfaces of the blood vessel contrast enhancement particle 10 and cancer contrast enhancement particle 20 by chemical modification. The PEGs 14 and 24 prevent the blood vessel contrast enhancement particle 10 and the cancer contrast enhancement particle 20 from being bonded to the surface proteins of vascular endothelial cells. The PEGs 14 and 24 need not always be chemically modified on both the blood vessel contrast enhancement particle 10 and the cancer contrast enhancement particle 20 and may be chemically modified on only one of them.

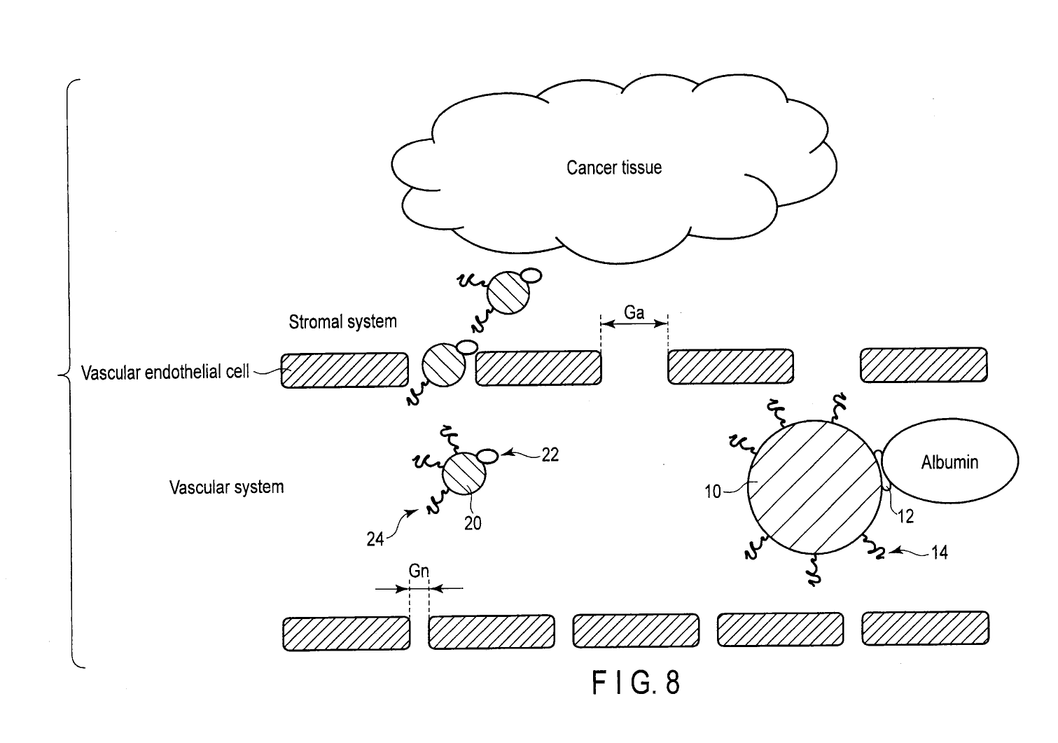

FIG. 8 is a view schematically showing the behaviors of the blood vessel contrast enhancement particle 10 and cancer contrast enhancement particle 20 provided with various modifications described above at the time of the occurrence of the EPR effect. As shown in FIG. 8, the blood vessel contrast enhancement particle 10 chemically modified with the PEG 14 and the cancer contrast enhancement particle 20 chemically modified with the PEG 24 can flow in the blood vessel for a long period of time without being bonded to surface proteins of vascular endothelial cells. Albumin existing in the blood vessel is bonded to the functional group 12 of the blood vessel contrast enhancement particle 10. The blood vessel contrast enhancement particle 10 to which the albumin is bonded is suppressed from being discharged from the blood vessel by the kidney, and can be retained in the blood vessel for a long period of time. In addition, chemically modifying the blood vessel contrast enhancement particle 10 with the PEG 14 can improve the fluidity of the blood vessel contrast enhancement particle 10 in the blood vessel. Furthermore, the ligand 22 of the cancer contrast enhancement particle 20 passing through the vascular endothelial cell gap Ga is specifically bonded to the receptor of a cancer cell. This makes it possible to specifically accumulate the cancer contrast enhancement particle 20 in the cancer cell. In addition, chemically modifying the cancer contrast enhancement particle 20 with the PEG 24 can synergistically increase the amount of cancer contrast enhancement particles 20 accumulated in the cancer tissue.

Human blood includes blood cells and blood plasma. Blood cells include erythrocytes, leukocytes, and platelets. Erythrocytes occupy most of the volume of blood cells. In general, an erythrocyte has a diameter of several μm, and a leukocyte has a diameter of ten several μm. As described above, the diameter of the cancer contrast enhancement particle 20 is much smaller than those of an erythrocyte and leukocyte, and the diameter of the blood vessel contrast enhancement particle 10 is between that of the cancer contrast enhancement particle 20 and those of an erythrocyte and a leukocyte. If only the cancer contrast enhancement particles 20 are injected into the object, the cancer contrast enhancement particles 20 are pushed back by blood cells such as erythrocytes and hence are difficult to pass through the vascular endothelial cell gaps. In contrast, when both the blood vessel contrast enhancement particles 10 and the cancer contrast enhancement particles 20 are injected into the object, the cancer contrast enhancement particles 20 can pass through the vascular endothelial cell gaps more efficiently, owing to a hydrodynamic effect, than when the blood vessel contrast enhancement particles 10 are not injected. Therefore, making the contrast agent according to this embodiment contain both the blood vessel contrast enhancement particles 10 and the cancer contrast enhancement particles 20 will improve the linearity between the amount of cancer contrast enhancement particles 20 injected into the blood vessel and the amount of cancer contrast enhancement particles 20 passing through the vascular endothelial cell gaps as compared with a case in which the contrast agent contains only the cancer contrast enhancement particles 20. In other words, the quantitativeness of the contrast enhancement effect of the stromal system by the cancer contrast enhancement particle 20 improves.

In addition, the blood vessel contrast enhancement particle 10 and the cancer contrast enhancement particle 20 may respectively contain materials having different contrast enhancement effects in the imaging principle of a modality to be used for imaging of the contrast agent according to this embodiment. Materials exhibiting dominant contrast enhancement effects in the respective contrast enhancement particles 10 and 20 will be referred to as contrast enhancement materials hereinafter. Since the blood vessel contrast enhancement particle 10 and the cancer contrast enhancement particle 20 respectively contain different contrast enhancement materials, the blood vessel contrast enhancement particle 10 and the cancer contrast enhancement particle 20 are depicted with different contrasts on the medical image generated by the modality. This allows the user to visually discriminate the blood vessel contrast enhancement particle 10 from the cancer contrast enhancement particle 20 on the medical image. That is, the contrast agent according to the embodiment can individually target the vascular system and stromal system of a cancer tissue and image the vascular system and the stromal system so as to make them visually discriminable for a long period of time.

The blood vessel contrast enhancement particle 10 and the cancer contrast enhancement particle 20 will be described in detail next. The blood vessel contrast enhancement particle 10 and the cancer contrast enhancement particle 20 will be referred to as contrast enhancement particles hereinafter unless they are discriminated from each other. A contrast enhancement particle includes a contrast enhancement material and a carrier whose particle size is set in the above manner. The contrast enhancement material is contained in the carrier or bonded to its surface and is carried to a target by the carrier. Typically, a carrier is formed from a material lower in contrast enhancement effect than a contrast enhancement material. As the carrier according to this embodiment, it is suitable to use a nanoparticle such as a liposome, polymer micelle, or dendrimer.

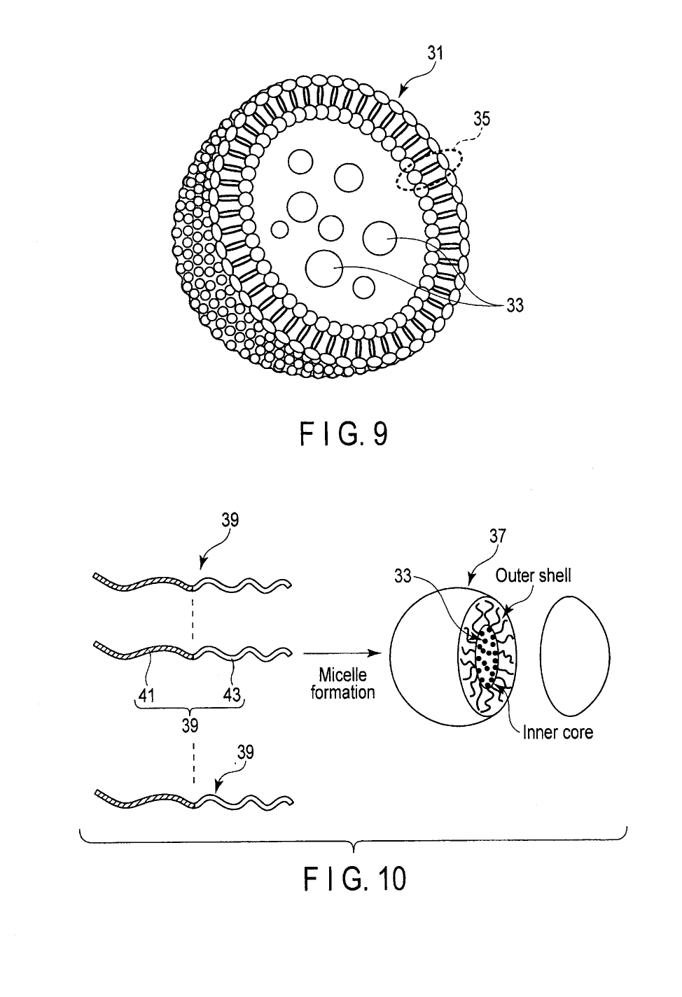

FIG. 9 is a view schematically showing a contrast enhancement particle when a carrier is a liposome 31. As shown in FIG. 9, the liposome 31 is a hollow vesicle composed of a spherically formed lipid bilayer membrane. Contrast enhancement materials 33 having a contrast enhancement effect implemented by a modality are contained in the liposome 31. The particle size of the liposome 31 can be adjusted by increasing/decreasing the number of phospholipids 35 constituting the lipid bilayer membrane. Although the liposome 31 is composed of the single lipid bilayer membrane in FIG. 9, the liposome may be composed of a plurality of lipid bilayer membranes. When the liposome 31 is the blood vessel contrast enhancement particle 10, the functional group 12 and the PEG 14 are formed on the surface of the liposome 31. When the liposome 31 is the cancer contrast enhancement particle 20, the ligand 22 and the PEG 24 are formed on the surface of the liposome 31. Note that the contrast enhancement materials 33 may be bonded to the surface of the liposome 31.

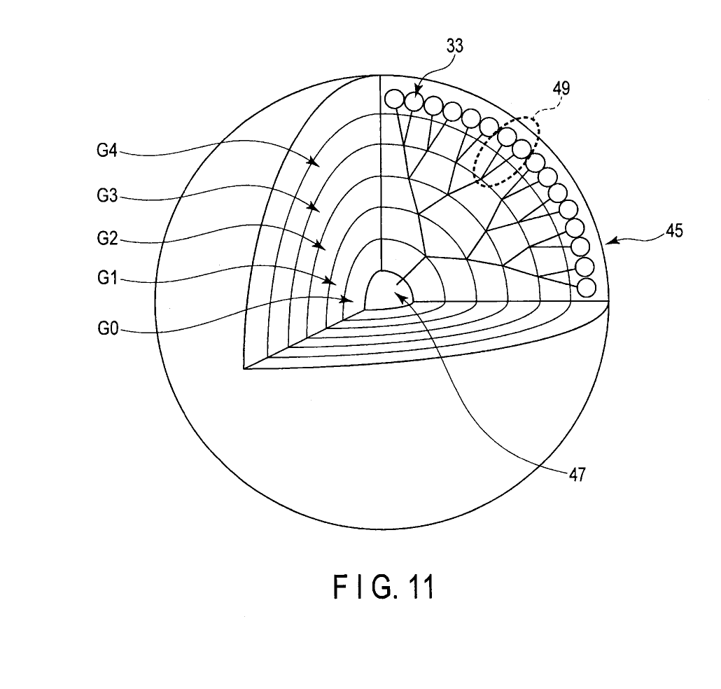

FIG. 10 is a view schematically showing a contrast enhancement particle when a carrier is a polymer micelle 37. As shown in FIG. 10, the polymer micelle 37 is a colloidal particle composed of a plurality of amphipathic block copolymers 39. Each block copolymer 39 contains a hydrophobic segment 41 and a hydrophilic segment 43. The polymer micelle 37 is formed from the plurality of block copolymers 39 such that the hydrophobic segments 41 of the plurality of block copolymers 39 form an inner core, and the hydrophilic segments 43 form an outer shell. The contrast enhancement material 33 may be chemically bonded to the hydrophobic segment 41 or physically adsorbed by the hydrophilic segment 43. As shown in FIG. 10, when chemically bonded to the hydrophobic segment 41, the contrast enhancement material 33 is contained in the polymer micelle 37 so as to be located in the inner core. Although not shown in FIG. 10, when physically adsorbed by the hydrophilic segment 43, the contrast enhancement material 33 is bonded to the polymer micelle 37 so as to be located on its surface. When the polymer micelle 37 is the blood vessel contrast enhancement particle 10, the functional group 12 and the PEG 14 are formed on the surface of the polymer micelle 37. When the polymer micelle 37 is the cancer contrast enhancement particle 20, the ligand 22 and the PEG 24 are formed on the surface of the polymer micelle 37. The particle size of the polymer micelle 37 can be adjusted by, for example, increasing/decreasing the length or number of block copolymers 39. For the sake of easy understanding, FIG. 10 shows the spherical outer shell. In practice, however, no spherical outer shell exists.

FIG. 11 is a view schematically showing a contrast enhancement particle when a carrier is a dendrimer 45. As shown in FIG. 11, the dendrimer 45 is composed of a plurality of unit molecular structures (side chains or dendrons) 49 extending from a central nucleus 47 and bonded to each other in a tree form. As the central nucleus 47, an atom having no contrast enhancement effect is preferably used. The number of times of branching from the central nucleus 47 to the dendrons 49 at the terminals are called generations. FIG. 11 exemplarily shows the dendrimer 45 composed of five generations from a 0th generation G0 to a fourth generation G4. However, the number of generations of the dendrimer 45 according to this embodiment is not limited to 5, and may be any number equal to or more than 1. The contrast enhancement materials 33 are bonded to the functional groups at the terminals of the dendrons 49, of the plurality of the dendrons 49, which are located on the surface side. When the dendrimer 45 is the blood vessel contrast enhancement particle 10, the functional group 12 and the PEG 14 are formed on the surface of the dendrimer 45. When the dendrimer 45 is the cancer contrast enhancement particle 20, the ligand 22 and the PEG 24 are formed on the surface of the dendrimer 45. The particle size of the dendrimer 45 can be adjusted by increasing/decreasing the number of generations, i.e., the number of times of branching of the dendrons 49. Note that FIG. 11 shows the spherical outer shell for the sake of easy understanding, no spherical outer shell practically exists.

According to the above description, the carrier according to this embodiment is a liposome, polymer micelle, or dendrimer. However, the carrier according to the embodiment is not limited to this. The carrier according to the embodiment may be any type of nanoparticle other than a liposome, polymer micelle, and dendrimer as long as a contrast enhancement material can be carried.

In addition, the blood vessel contrast enhancement particle 10 and the cancer contrast enhancement particle 20 may contain the same or different types of carriers. It is possible to individually select optimal types of carriers from the viewpoint of the reliability, easiness, and the like of transfer, accumulation, and retention of the contrast enhancement particles 10 and 20 with respect to a contrast enhancement target (target).

A specific example of the contrast agent according to this embodiment will be described for each type of modality. The types of modalities according to the embodiment are classified into single modalities and composite modalities. As a single modality according to the embodiment, any one of a PCCT (photon counting CT) apparatus, X-ray computed tomographic apparatus, X-ray diagnostic apparatus, PET apparatus, and SPECT (single photon emission CT) apparatus, and MRI (magnetic resonance imaging) apparatus can be used. As a composite modality according to the embodiment, a combination of any of the above modalities can be used. A suitable composite modality according to the embodiment includes for example, a PCCT/CT apparatus, PET/CT apparatus, SPECT/CT apparatus, and PET/MRI apparatus. A specific example of a contrast agent for each modality will be described below.

### (Single Modality)

When the contrast agent according to this embodiment is to be used for imaging by a single modality, contrast enhancement materials having almost the same contrast enhancement effect in the imaging principle of the single modality are selected as those for the blood vessel contrast enhancement particle 10 and the cancer contrast enhancement particle 20. In this case, it is preferable to select materials from which almost the same contrast can be obtained by the same contrast enhancement mechanism, as needed, as contrast enhancement materials for the blood vessel contrast enhancement particle 10 and the cancer contrast enhancement particle 20. Alternatively, contrast enhancement materials having different contrast enhancement effects in the imaging principle of the single modality are preferably selected as those for the blood vessel contrast enhancement particle 10 and the cancer contrast enhancement particle 20. In other words, materials from which different contrasts can be obtained by the same contrast enhancement mechanism are selected, as needed, as contrast enhancement materials for the blood vessel contrast enhancement particle 10 and the cancer contrast enhancement particle 20.

PCCT Apparatus: for example, a PCCT apparatus irradiates an object with X-rays from an X-ray tube while rotating the X-ray tube and an X-ray detector around the object, detects the X-rays transmitted through the object by using the X-ray detector, and counts the number of detected X-ray photons for each energy band. The PCCT apparatus generates an image expressing the spatial distribution of the numbers of photons for each energy band. The contrast enhancement mechanism of a contrast agent for PCCT (to be referred to as PCCT contrast agent hereinafter) can change the intensity of X-ray photons transmitted through the contrast agent by increasing the X-ray attenuation coefficient difference between the contrast agent and a surrounding tissue. As a contrast enhancement material for a PCCT contrast agent, it is preferable to use a heavy metal or the like higher in X-ray attenuation coefficient than a surrounding tissue of a contrast enhancement target. Such heavy metals include, for example, iodine I, gadolinium Gd, gold Au, and bismuth Bi. If the blood vessel contrast enhancement particle 10 and the cancer contrast enhancement particle 20 need not have any contrast difference, similar types of heavy metals are preferably selected, as needed, from the heavy metals such as iodine I, gadolinium Gd, gold Au, and bismuth Bi as the contrast enhancement materials for the blood vessel contrast enhancement particle 10 and the cancer contrast enhancement particle 20. If the blood vessel contrast enhancement particle 10 and the cancer contrast enhancement particle 20 need to have a contrast difference, different types of heavy metals having different contrast enhancement effects are preferably selected, as needed, from the heavy metals such as iodine I, gadolinium Gd, gold Au, and bismuth Bi as the contrast enhancement materials for the blood vessel contrast enhancement particle 10 and the cancer contrast enhancement particle 20. As a carrier, it is possible to use any of a liposome, polymer micelle, and dendrimer which can contain a heavy metal having a high X-ray attenuation coefficient or to which it can be bonded. Note that gadolinium Gd, gold Au, and bismuth Bi have slight toxicity to the human body, whereas a liposome has the property capable of reducing toxicity. For this reason, when gadolinium Gd, gold Au, or bismuth Bi is to be used as a contrast enhancement material, the contrast enhancement material is preferably contained in a liposome. Note that if it is possible to reduce toxicity by a technique other than being contained in a liposome, gadolinium Gd, gold Au, or bismuth Bi may be contained in or bonded to any carrier.

X-ray Computed Tomographic Apparatus: for example, an X-ray computed tomographic apparatus irradiates an object with X-rays from an X-ray tube while rotating the X-ray tube and an X-ray detector around the object, and detects the X-rays transmitted through the object by using the X-ray detector. The X-ray computed tomographic apparatus generates an image expressing the spatial distribution of the X-ray attenuation coefficients of substances on the X-ray transmission path. The contrast enhancement mechanism of a contrast agent for X-ray CT (to be referred to as a CT contrast agent hereinafter) can change the intensity of X-rays transmitted through the contrast agent by increasing the X-ray attenuation coefficient difference between the contrast agent and a surrounding tissue. As a contrast enhancement material for the CT contrast agent, it is preferable to use a heavy metal having a high X-ray attenuation coefficient such as iodine I. As a carrier, it is possible to use any of a liposome, polymer micelle, and dendrimer which can contain a heavy metal having a high X-ray attenuation coefficient or to which it can be bonded.

X-ray Diagnostic Apparatus: an X-ray diagnostic apparatus according to this embodiment may be of a current integration type or photon counting type. The X-ray diagnostic apparatus of the current integration type irradiates an object with X-rays from an X-ray tube at a desired imaging angle, and detects the X-rays transmitted through the object by using an X-ray detector. The X-ray diagnostic apparatus of the current integration type generates an image expressing the spatial distribution of the X-ray attenuation coefficients of substances on the X-ray transmission path. The contrast enhancement mechanism of a contrast agent in the current integration type is similar to that of a CT contrast agent. As a contrast enhancement material for the contrast agent in the current integration type, it is preferable to use a heavy metal having a high X-ray attenuation coefficient such as iodine I. As a carrier, it is possible to use any of liposome, polymer micelle, and dendrimer which can contain a heavy metal having an X-ray attenuation coefficient or to which it can be bonded. The X-ray diagnostic apparatus of the photon counting type irradiates an object with X-rays from an X-ray tube at a desired imaging angle, detects the X-rays transmitted through the object by using an X-ray detector, and counts the number of detected X-ray photons for each energy band. The X-ray diagnostic apparatus of the photon counting type generates an image expressing the spatial distribution of the numbers of photons for each energy band. The contrast enhancement mechanism of a contrast agent in the photon counting type is similar to that of a PCCT contrast agent. As a contrast enhancement material for a contrast agent in the photon counting type, it is preferable to use a heavy metal or the like having a high X-ray attenuation coefficient, such as iodine I, gadolinium Gd, gold Au, and bismuth Bi. If the blood vessel contrast enhancement particle 10 and the cancer contrast enhancement particle 20 need not have any contrast difference, similar types of heavy metals are preferably selected, as needed, from the heavy metals such as iodine I, gadolinium Gd, gold Au, and bismuth Bi as the contrast enhancement materials for the blood vessel contrast enhancement particle 10 and the cancer contrast enhancement particle 20. If the blood vessel contrast enhancement particle 10 and the cancer contrast enhancement particle 20 need to have a contrast difference, different types of heavy metals having different contrast enhancement effects are preferably selected, as needed, from the heavy metals such as iodine I, gadolinium Gd, gold Au, and bismuth Bi as the contrast enhancement materials for the blood vessel contrast enhancement particle 10 and the cancer contrast enhancement particle 20. As a carrier, it is possible to use any of a liposome, polymer micelle, and dendrimer which can contain a heavy metal having a high X-ray attenuation coefficient or to which it can be bonded. Note that gadolinium Gd, gold Au, and bismuth Bi have slight toxicity to the human body, whereas a liposome has the property capable of reducing toxicity. For this reason, when gadolinium Gd, gold Au, or bismuth Bi is to be used as a contrast enhancement material, the contrast enhancement material is preferably contained in a liposome. Note that if it is possible to reduce toxicity by a technique other than being contained in a liposome, gadolinium Gd, gold Au, or bismuth Bi may be contained in or bonded to any carrier.

PET apparatus: a PET apparatus simultaneously measures a pair of 512-keV gamma rays generated upon pair annihilation of each of positrons generated from radionuclides accumulated in an object and a corresponding one of electrons existing around the radionuclides, thereby generating an image expressing the spatial density distribution of the radionuclides. A contrast enhancement material for PET imaging may be any type of radionuclide which can emit positrons. It is preferable to use, as a contrast enhancement material for PET imaging, ¹⁸F which is the radionuclide of fluorine, ¹¹C which is the radionuclide of carbon, or the like. As a carrier, it is possible to use any of a liposome, polymer micelle, and dendrimer which can contain the radionuclide. When the contrast enhancement material is to be contained in a liposome, the contrast enhancement material is preferably contained in the liposome upon being synthesized with a suitable compound for the purpose of, for example, a reduction in the toxicity of the contrast enhancement material. When the contrast enhancement material is to be bonded to the surface of a liposome, the contrast enhancement material is preferably bonded to the surface of the liposome upon being synthesized with a suitable compound for the purpose of, for example, a reduction in the toxicity of the contrast enhancement material. When the contrast enhancement material is to be contained in a polymer micelle, the contrast enhancement material is contained in the polymer micelle upon being reactively synthesized with the hydrophobic segment of a block copolymer. When using a dendrimer, the contrast enhancement material is bonded to the functional group at the terminal of a dendron.

SPECT Apparatus: A SPECT apparatus detects single photon gamma rays generated from radionuclides accumulated in an object to generate an image expressing the spatial density distribution of the radionuclides. A contrast enhancement material for SPECT imaging may be any type of radionuclide which can emit single photon gamma rays. It is preferable to use, as a contrast enhancement material for SPECT imaging, ^{99m}TC which is the radionuclide of technetium, ²⁰¹Tl which is the radionuclide of thallium, or the like. The energy of a single photon gamma ray varies in accordance with the type of radionuclide which emits the single photon gamma ray. As a carrier, it is possible to use any of a liposome, polymer micelle, and dendrimer which can contain the radionuclide. When the contrast enhancement material is to be contained in a liposome, the contrast enhancement material is preferably contained in the liposome upon being synthesized with a suitable compound for the purpose of, for example, a reduction in the toxicity of the contrast enhancement material. When the contrast enhancement material is to be bonded to the surface of a liposome, the contrast enhancement material is preferably bonded to the surface of the liposome upon being synthesized with a suitable compound for the purpose of, for example, a reduction in the toxicity of the contrast enhancement material. When the contrast enhancement material is to be contained in a polymer micelle, the contrast enhancement material is contained in the polymer micelle upon being reactively synthesized with the hydrophobic segment of a block copolymer. When using a dendrimer, the contrast enhancement material is bonded to the functional group at the terminal of a dendron.

MRI Apparatus: an MRI apparatus can use a plurality of imaging principles in accordance with imaging purposes. The imaging principles include, for example, that for an imaging operation using the difference in a longitudinal relaxation time T1 or a transverse relaxation time T2 and that for an imaging operation using the CEST (Chemical Exchange Saturation Transfer) effect. Different types of contrast enhancement materials are used in accordance with different imaging principles.

1. In imaging using the difference in T1 or T2, a contrast enhancement material having the effect of shortening T1 or T2 is used. As such a contrast enhancement material, a paramagnetic metal, SPIO (Superparamagnetic iron oxide particle), or the like is used. As a paramagnetic metal, gadolinium Gd or manganese Mn is used. If the blood vessel contrast enhancement particle 10 and the cancer contrast enhancement particle 20 need not have any contrast difference, similar types of metals are preferably selected from the above paramagnetic metals and superparamagnetic iron oxide particles as the contrast enhancement materials for the blood vessel contrast enhancement particle 10 and the cancer contrast enhancement particle 20. If the blood vessel contrast enhancement particle 10 and the cancer contrast enhancement particle 20 need to have a contrast difference, different types of metals having different contrast enhancement effects are preferably selected, as needed, from the above paramagnetic metals and superparamagnetic iron oxide particles as the contrast enhancement materials for the blood vessel contrast enhancement particle 10 and the cancer contrast enhancement particle 20. Note that as contrast enhancement materials for the blood vessel contrast enhancement particle 10 and the cancer contrast enhancement particle 20, contrast enhancement materials having different resonant frequencies instead of contrast enhancement materials having the effect of shortening T1 or T2 may be used.

2. In imaging using the CEST effect (to be referred to as CEST imaging hereinafter), a compound containing a paramagnetic metal which can be an exogenous contrast agent is used as a contrast enhancement material. A compound containing a paramagnetic metal aimed at CEST imaging is called a PARACEST contrast agent. As this paramagnetic metal, it is preferable to use a paramagnetic metal belonging to the lanthanoid elements including europium Eu, terbium Tb, dysprosium Dy, ytterbium Yb, and thulium Tm. If the blood vessel contrast enhancement particle 10 and the cancer contrast enhancement particle 20 need not have any contrast difference, similar types of metals are preferably selected, as needed, from the above paramagnetic metals belonging to the lanthanoid elements as paramagnetic metals for the blood vessel contrast enhancement particle 10 and the cancer contrast enhancement particle 20. If the blood vessel contrast enhancement particle 10 and the cancer contrast enhancement particle 20 need to have a contrast difference, metals having different contrast enhancement effects are preferably selected, as needed, from the above paramagnetic metals belonging to the lanthanoid elements as the contrast enhancement materials for the blood vessel contrast enhancement particle 10 and the cancer contrast enhancement particle 20. The mechanism of an endogenous contrast agent will be described as an explanation of a general CEST effect. When the proton H (for example, the proton H of —NH₂ (amide group)) contained in an endogenous contrast agent is continuously excited at a resonant frequency corresponding to the proton, magnetic energy is exchanged between a proton of the endogenous contrast agent and a proton of free water according to the chemical exchange phenomenon. This magnetic energy exchange will decrease the intensity of MR signals from the protons of the free water. This is a phenomenon called the CEST effect. In an exogenous contrast agent (PARACEST contrast agent), direct magnetic energy exchange between the protons of free water and the paramagnetic metal in the exogenous contrast agent will decrease the intensity of MR signals from the protons of the free water as described above. It is known that the CEST imaging is higher in detection sensitivity than conventional MR spectroscopy by at least 10 times or more.

When a liposome is to be used as a carrier, a contrast enhancement material is preferably contained in the liposome or bonded to it surface. If a contrast enhancement material has toxicity, since a lipid bilayer membrane itself has a structure which reduces the toxicity, the contrast enhancement material is preferably contained in the liposome. In order to further reduce the toxicity, the contrast enhancement material may be synthesized into a compound having a chelate structure by using a chelator, and the contrast enhancement material having the chelate structure may be contained in the liposome. Alternatively, the contrast enhancement material may be synthesized with a simpler compound which can reduce the toxicity, and the synthetic compound may be contained in the liposome. When a polymer micelle is to be used as a carrier, a contrast enhancement material having the above chelate structure is preferably reacted with a block copolymer, and the contrast enhancement material having a chelate structure is preferably contained in the polymer micelle. When a dendrimer is to be used as a carrier, the contrast enhancement material having a chelate structure is bonded to the functional group at the terminal of a dendron.

Ultrasonic Diagnostic Apparatus: an ultrasonic diagnostic apparatus generates an image expressing the spatial distribution of acoustic impedance differences of substances existing in an object by receiving reflected waves of ultrasonic waves transmitted into the object. The contrast enhancement mechanism of a contrast agent in ultrasonic imaging (to be referred to as an ultrasonic contrast agent hereinafter) can increase the reflection intensity of ultrasonic waves using the contrast agent by increasing the acoustic impedance differences between the contrast agent and a surrounding tissue. It is therefore not necessary to use any contrast enhancement material for the ultrasonic contrast agent. As a carrier for the ultrasonic contrast agent, it is possible to use a nanoparticle such as a liposome, polymer micelle, or dendrimer. However, as a carrier for the ultrasonic contrast agent, it is preferable to use spherical nanoparticles which can isotropically reflect ultrasonic waves. From this point of view, a liposome is suitable as a carrier for the ultrasonic contrast agent. Ultrasonic contrast agents are roughly classified into first and second generations. A first-generation ultrasonic contrast agent is used to visualize the behavior of the ultrasonic contrast agent accumulated in a target, which is perfused to the target again after being crushed by ultrasonic waves. For this reason, as the first-generation ultrasonic contrast agent, a hollow liposome is suitably used. The type of gas to be contained in the liposome is not specifically limited. The gas to be contained in the liposome includes, for example, air and hydrogen fluoride. A second-generation ultrasonic contrast agent is used to visualize the behavior of the scattered ultrasonic contrast agent which has been accumulated in a target and scattered by ultrasonic waves. For this reason, the second-generation ultrasonic contrast agent need not be hollow, and any type of nanoparticle such as a liposome, polymer micelle, or dendrimer can be used. Even when a carrier such as a liposome, polymer micelle, or dendrimer is to be used as an ultrasonic contrast agent, the functional group 12, the PEG 14, the ligand 22, and the PEG 24 can be bonded to the surface of the carrier, as needed.

### (Composite Modality)

When the contrast agent according to this embodiment is to be used for imaging by a composite modality, a material exhibiting a contrast enhancement effect in the imaging principle of the first modality of the composite modality is selected as the contrast enhancement material for the blood vessel contrast enhancement particle 10, and a material exhibiting a contrast enhancement effect in the imaging principle of the second modality of the composite modality is selected as the contrast enhancement material for the cancer contrast enhancement particle 20. In other words, a material from which a contrast can be obtained by a contrast enhancement mechanism based on the imaging principle of the first modality is used as the contrast enhancement material for the blood vessel contrast enhancement particle 10, and a material from which a contrast can be obtained by a contrast enhancement mechanism based on the imaging principle of the second modality is used as the contrast enhancement material for the cancer contrast enhancement particle 20.

PCCT/CT Apparatus: a PCCT/CT apparatus is a composite apparatus constituted by a PCCT apparatus and an X-ray computed tomographic apparatus. The X-ray computed tomographic apparatus preferably performs vascular system imaging. The PCCT apparatus preferably performs stromal system imaging. For this reason, iodine is preferably used as the contrast enhancement material for the blood vessel contrast enhancement particle 10. In addition, as the contrast enhancement material for the cancer contrast enhancement particle 20, one of heavy metals including iodine I, gadolinium Gd, gold Au, and bismuth Bi is preferably selected as needed. Note that according to the above description, the X-ray computed tomographic apparatus performs vascular system imaging, and the PCCT apparatus performs stromal system imaging. However, this embodiment is not limited to this. That is, the PCCT apparatus may perform vascular system imaging, and the X-ray computed tomographic apparatus may perform stromal system imaging.

PET/CT Apparatus: a PET/CT apparatus is a composite apparatus constituted by a PET apparatus and an X-ray computed tomographic apparatus. The X-ray computed tomographic apparatus preferably performs vascular system imaging. The PET apparatus preferably performs stromal system imaging. For this reason, iodine I is preferably used as the contrast enhancement material for the blood vessel contrast enhancement particle 10. The above radionuclide which can emit positrons is preferably used as the contrast enhancement material for the cancer contrast enhancement particle 20. Note that according to the above description, the X-ray computed tomographic apparatus performs vascular system imaging, and the PET apparatus performs stromal system imaging. However, this embodiment is not limited to this. That is, the PET apparatus may perform vascular system imaging, and the X-ray computed tomographic apparatus may perform stromal system imaging.

SPECT/CT Apparatus: a SPECT/CT apparatus is a composite apparatus constituted by a SPECT apparatus and an X-ray computed tomographic apparatus. The X-ray computed tomographic apparatus preferably performs vascular system imaging. The SPECT apparatus preferably performs stromal system imaging. For this reason, iodine I is preferably used as the contrast enhancement material for the blood vessel contrast enhancement particle 10. The above radionuclide which can emit single photon gamma rays is preferably used as the contrast enhancement material for the cancer contrast enhancement particle 20. Note that according to the above description, the X-ray computed tomographic apparatus performs vascular system imaging, and the SPECT apparatus performs stromal system imaging. However, this embodiment is not limited to this. That is, the SPECT apparatus may perform vascular system imaging, and the X-ray computed tomographic apparatus may perform stromal system imaging.

PET/MRI Apparatus: a PET/MRI apparatus is a composite apparatus constituted by a PET apparatus and an MRI apparatus. The MRI apparatus preferably performs vascular system imaging. The PET apparatus preferably performs stromal system imaging. More specifically, vascular system imaging is preferably performed by MR angiography which depicts a contrast-enhanced blood vessel by performing imaging using the difference in T1 or T2. Therefore, a paramagnetic metal or superparamagnetic iron oxide particle is preferably used as the contrast enhancement material for the blood vessel contrast enhancement particle 10. In addition, the above radionuclide which can emit positrons is preferably used as the contrast enhancement material for the cancer contrast enhancement particle 20. Note that according to the above description, the MRI apparatus performs vascular system imaging, and the PET apparatus performs stromal system imaging. However, this embodiment is not limited to this. That is, the PET apparatus may perform vascular system imaging, and the MRI apparatus may perform stromal system imaging. In the above description, vascular system imaging is performed by MR angiography. However, this embodiment is not limited to this. In addition, when stromal system imaging is to be performed by the MRI apparatus, CEST imaging may be used. In this case, as the contrast enhancement material for the cancer contrast enhancement particle 20, it is preferable to use a paramagnetic metal belonging to the lanthanoid elements.

As described above, the contrast agent according to this embodiment contains the blood vessel contrast enhancement particle 10 for enhancing the contrast of a blood vessel of an object and the diseased tissue contrast enhancement particle (the cancer tissue contrast enhancement particle when a contrast enhancement target is a cancer tissue) 20 for enhancing the contrast of a diseased tissue such as a cancer tissue of the object. The blood vessel contrast enhancement particle 10 has a particle size larger than the vascular endothelial cell gap Ga under the EPR effect, and diseased tissue contrast enhancement particle 20 has a particle size smaller than the gap Ga. The blood vessel contrast enhancement particle 10 and the diseased tissue contrast enhancement particle 20 contain contrast enhancement materials having different contrast enhancement effects.

When the contrast agent according to this embodiment is injected into a blood vessel of an object, the diseased tissue contrast enhancement particles 20 pass through the vascular endothelial cell gaps Ga of a neighboring blood vessel or new nutrient vessel for a diseased tissue and are accumulated in the diseased tissue. The blood vessel contrast enhancement particles 10 have a particle size larger than the gaps Ga, and hence are retained in the blood vessel because of incapability to pass through the gaps Ga. Imaging the object by using a single modality upon injection of the contrast agent according to the embodiment can acquire an image (to be referred to as stromal system/vascular system image hereinafter) depicting both the stromal system and vascular system of the diseased tissue so as to make them visually discriminable. A stromal system/vascular system image is displayed on a display device by a single modality or the like. A user such as a technician observes the displayed stromal system/vascular system image. The user can accurately grasp the states of the stromal system and vascular system of the diseased tissue from one image (stromal system/vascular system image). It is possible to obtain an image which is excellent, in particular, in quantitativeness.

In addition, it is possible to individually acquire an image (to be referred to as a stromal system image hereinafter) clearly depicting a stromal system as compared with a vascular system and an image (to be referred to as a vascular system image hereinafter) clearly depicting a vascular system as compared with a stromal system by imaging an object a plurality of times at different timings using a single modality. An imaging timing can be arbitrarily decided in accordance with an instruction issued by the user via an input device of the medical image diagnostic apparatus. A stromal system image and a vascular system image may be individually acquired by imaging an object at the same timing or different timings using a composite modality. More specifically, if two modalities constituting a composite modality can simultaneously image the same region, the first modality performs imaging of the stromal system, and the second modality performs imaging of the vascular system. Composite modalities capable of simultaneously imaging include, for example, a PET/MRI apparatus. If two modalities constituting a composite modality cannot simultaneously image the same region, the first and second modalities may respectively perform imaging of the stromal system and imaging of the vascular system at different timings. The imaging timing of each modality can be arbitrarily decided by the user via an input device of the composite modality. A composite modality or the like displays a stromal system image and a vascular system image on a display device. A user such as a technician can individually grasp the states of the stromal system and vascular system of a diseased tissue by observing the stromal system image and the vascular system image. A stromal system image and a vascular system image may be displayed on the display device upon being positionally matched with each other. Using the contrast agent according to this embodiment makes it possible to perform simultaneous imaging or time-series imaging of the stromal system and vascular system of a diseased tissue.

### (Applications)

The blood vessel contrast enhancement particles 10 and the cancer contrast enhancement particles 20 according to this embodiment may be mixed at an arbitrary ratio. However, it is preferable to mix the blood vessel contrast enhancement particles 10 with the cancer contrast enhancement particles 20 at a ratio corresponding to the purpose of use of the contrast agent. In this case, the ratio at which the blood vessel contrast enhancement particles 10 are mixed with the cancer contrast enhancement particles 20 will be referred to as a mixing ratio. A mixing ratio is defined by the abundance of the diseased tissue contrast enhancement particles 20 in the entire contrast agent relative to the abundance of the blood vessel contrast enhancement particles 10 in the entire contrast agent. An abundance may be any amount including the weight, volume, or molar concentration of the blood vessel contrast enhancement particles 10 or cancer contrast enhancement particles 20.

For example, the contrast agent according to this embodiment is sometimes used to detect a treatment target. In this case, it is important to specify the presence or the like of a tumor nutrient blood vessel by vascular system imaging. For this reason, it is preferable to mix the blood vessel contrast enhancement particles 10 with the cancer contrast enhancement particles 20 such that the abundance of the blood vessel contrast enhancement particles 10 in the entire contrast agent is larger than that of the cancer contrast enhancement particles 20 in the entire contrast agent. For example, it is preferable to mix the blood vessel contrast enhancement particles 10 with the cancer contrast enhancement particles 20 at a mixing ratio of 2:1. A vascular system is enhanced compared with a stromal system on a medical image by imaging an object injected with the contrast agent containing the blood vessel contrast enhancement particles 10 and the cancer contrast enhancement particles 20 mixed at such a ratio. This allows the user to efficiently specify the presence or the like of a tumor nutrient blood vessel.

In addition, the contrast agent according to this embodiment is sometimes used to determine a treatment effect. In this case, since the main purpose is to observe a treatment process, stromal system imaging is more important than vascular system imaging. For this reason, it is preferable to mix the blood vessel contrast enhancement particles 10 with the cancer contrast enhancement particles 20 such that the abundance of the blood vessel contrast enhancement particles 10 in the entire contrast agent is smaller than that of the cancer contrast enhancement particles 20 in the entire contrast agent. For example, the blood vessel contrast enhancement particles 10 are preferably mixed with the cancer contrast enhancement particles 20 at a mixing ratio of 1:2. A stromal system is enhanced compared with a vascular system on a medical image by imaging an object injected with the contrast agent containing the blood vessel contrast enhancement particles 10 and the cancer contrast enhancement particles 20 mixed at such a ratio. This allows the user to efficiently specify a treatment effect on the stromal system.

### (Modification)

An ultrasonic contrast agent according to a modification of this embodiment will be described below. The blood vessel contrast enhancement particles and cancer contrast enhancement particles contained in the ultrasonic contrast agent according to this modification differ in ultrasonic intensity or frequency at which the particles can be crushed or excessively vibrated. In the following description, the same reference numerals denote constituent elements having almost the same functions as in the above embodiment, and a repetitive description will be made only when required. Although a phenomenon in which particles are crushed by ultrasonic waves will be described below, the same applies to particles which undergo excessive vibrations caused by ultrasonic waves. The following description will exemplify the case of using differences in the occurrence of a crushing phenomenon due to differences in ultrasonic frequency. However, it is possible to use differences in the occurrence of a crushing phenomenon due to differences in the transmission intensity of ultrasonic waves. In addition, it is possible to use differences in the occurrence of an excessive vibration phenomenon due to the transmission intensity of ultrasonic waves.

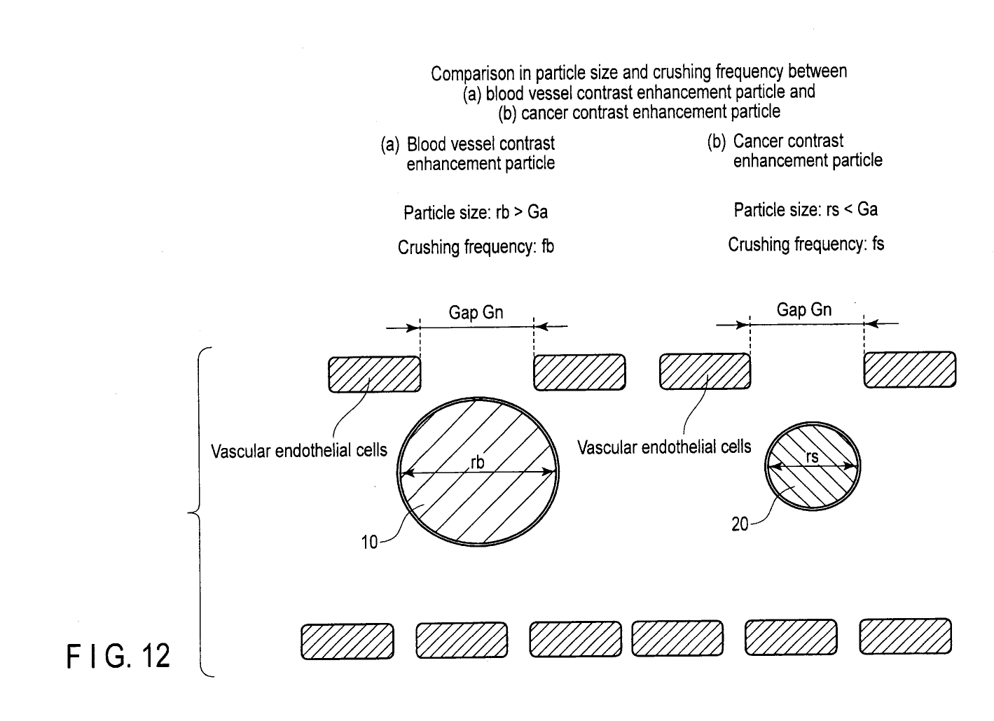

FIG. 12 is a view showing the comparisons between the particle size and crushing frequency of the blood vessel contrast enhancement particle 10 and those of the cancer contrast enhancement particle 20 contained in an ultrasonic contrast agent according to a modification. Assume that a carrier for the blood vessel contrast enhancement particle 10 and the cancer contrast enhancement particle 20 is a liposome. As shown in FIG. 12, the blood vessel contrast enhancement particle 10 is adjusted to be larger than the vascular endothelial cell gap Ga at the time of the occurrence of the EPR effect, and the cancer contrast enhancement particle 20 is adjusted to be smaller than the gap Ga. The strengths of the blood vessel contrast enhancement particle 10 and cancer contrast enhancement particle 20 are adjusted such that the respective particles are crushed by ultrasonic waves with different frequencies or intensities. For example, the structural strengths of the blood vessel contrast enhancement particle 10 and cancer contrast enhancement particle 20 are adjusted such that the blood vessel contrast enhancement particle 10 is crushed upon receiving an ultrasonic wave with a frequency higher than a frequency fb, and the cancer contrast enhancement particle 20 is crushed upon receiving an ultrasonic wave with a frequency higher than a frequency fs. The structure strengths of the blood vessel contrast enhancement particle 10 and cancer contrast enhancement particle 20 can be adjusted by various techniques. For example, it is preferable to adjust a structural strength by coating a liposome with a material such as carbon.

The lower limit of frequencies at which the blood vessel contrast enhancement particle 10 or the cancer contrast enhancement particle 20 can be crushed will be referred to as a crushing frequency hereinafter. The magnitude relationship in crushing frequency between the blood vessel contrast enhancement particle 10 and the cancer contrast enhancement particle 20 can be arbitrarily adjusted in accordance with a reperfusion observation target. If, for example, the reperfusion of the cancer contrast enhancement particle 20 to a cancer tissue is an observation target, the crushing frequency fs of the cancer contrast enhancement particle 20 is set to be lower than the crushing frequency of the blood vessel contrast enhancement particle 10. If the reperfusion of the blood vessel contrast enhancement particle 10 to a diseased blood vessel region is an observation target, the crushing frequency fb of the blood vessel contrast enhancement particle 10 is set to be lower than the crushing frequency fs of the cancer contrast enhancement particle 20. In other words, the crushing frequencies fb and fs for the contrast enhancement particles 10 and 20 to be crushed are adjusted to be lower than the frequencies of ultrasonic waves (to be referred to as crushing ultrasonic waves hereinafter) for crushing the contrast enhancement particles, whereas the crushing frequencies fb and fs for the contrast enhancement particles 10 and 20 not to be crushed are adjusted to be higher than the frequencies of crushing ultrasonic waves. This makes it possible to selectively crush the blood vessel contrast enhancement particle 10 and the cancer contrast enhancement particle 20.

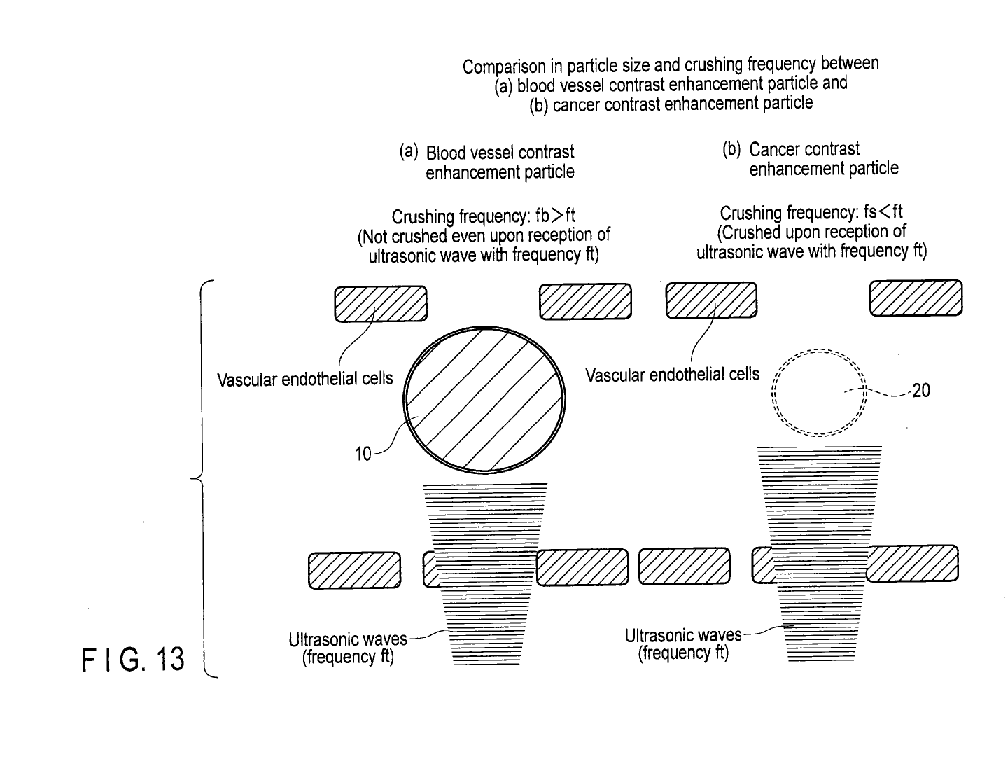

FIG. 13 is a view showing the comparison between a crushing frequency ft and the crushing frequencies fb and fs of the blood vessel contrast enhancement particle 10 and cancer contrast enhancement particle 20 in a case in which the reperfusion of the cancer contrast enhancement particle 20 into a cancer tissue is an observation target. As shown in FIG. 13, the crushing frequency fs for the cancer contrast enhancement particle 20 is set to be lower than the frequency ft of crushing ultrasonic waves. When, therefore, the cancer contrast enhancement particle 20 receives a crushing ultrasonic wave of the frequency ft, the cancer contrast enhancement particle 20 is crushed. In contrast, the crushing frequency fb for the blood vessel contrast enhancement particle 10 is set to be higher than the frequency ft to prevent the blood vessel contrast enhancement particle 10 from being crushed, together with the cancer contrast enhancement particle 20, by the application of an crushing ultrasonic wave of the frequency ft. It is possible to selectively crush the blood vessel contrast enhancement particle 10 and the cancer contrast enhancement particle 20 by setting the crushing frequency for only one of the blood vessel contrast enhancement particle 10 and the cancer contrast enhancement particle 20 to be lower than the frequency ft in this manner.

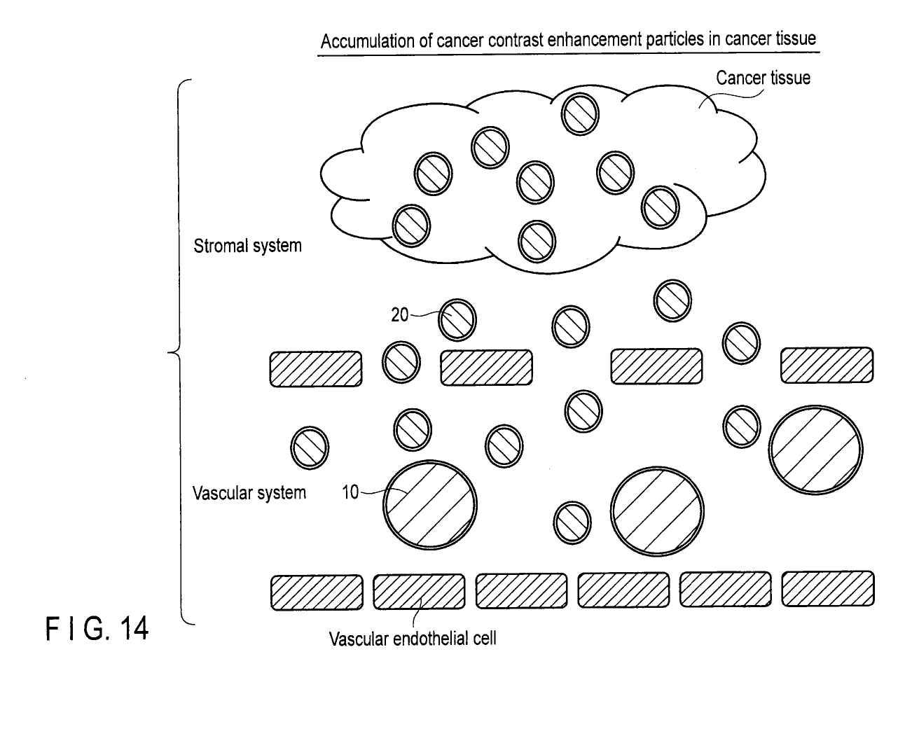

The behaviors of the blood vessel contrast enhancement particle 10 and cancer contrast enhancement particle 20 in ultrasonic imaging of the reperfusion of the cancer contrast enhancement particle 20 to a cancer tissue as an observation target will be described below with reference to FIGS. 14, 15, and 16.

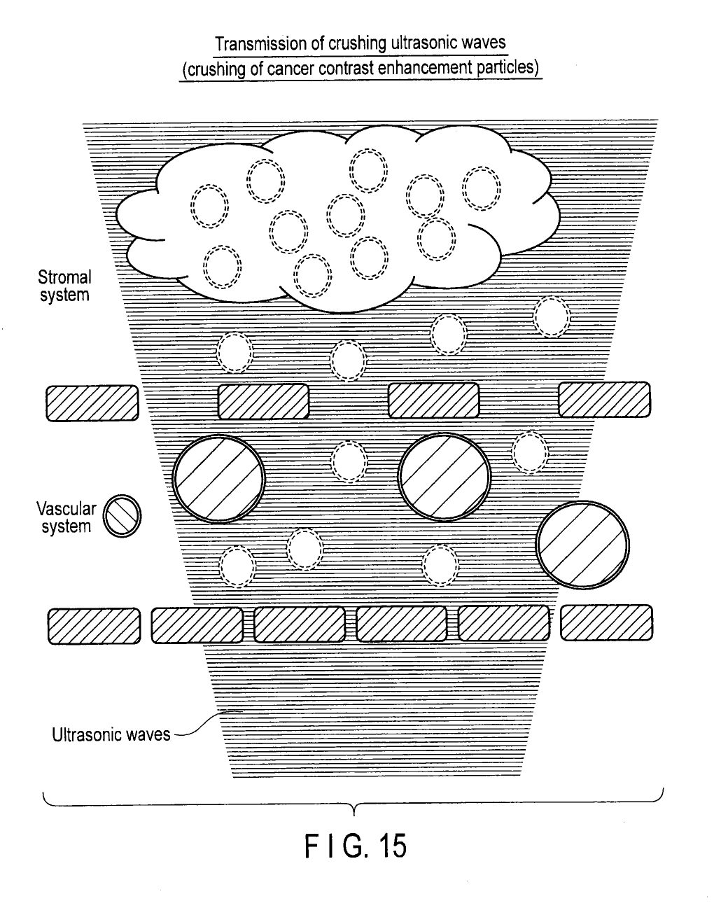

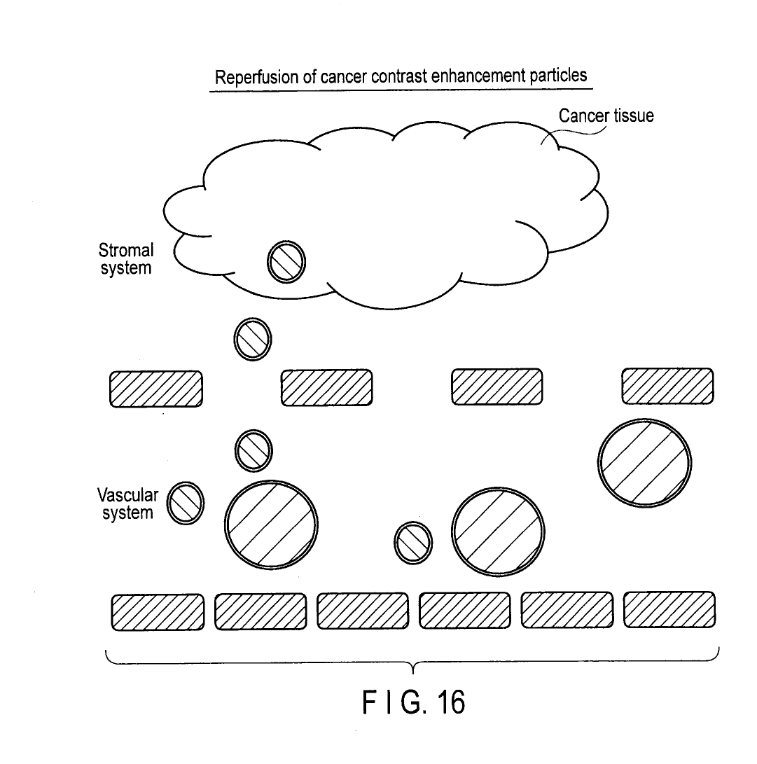

FIG. 14 is a view schematically showing the behaviors of the blood vessel contrast enhancement particles 10 and cancer contrast enhancement particles 20 in the stage of accumulation of the cancer contrast enhancement particles 20 in a cancer tissue. FIG. 15 is a view schematically showing the behaviors of the blood vessel contrast enhancement particles 10 and cancer contrast enhancement particles 20 in the stage of transmission of crushing ultrasonic waves. FIG. 16 is a view schematically showing the behaviors of the blood vessel contrast enhancement particles 10 and cancer contrast enhancement particles 20 in the stage of accumulation of the cancer contrast enhancement particles 20 in a cancer tissue.

As shown in FIG. 14, when the contrast agent according to this embodiment, which contains the blood vessel contrast enhancement particles 10 and the cancer contrast enhancement particles 20, is injected into a blood vessel, the blood vessel contrast enhancement particles 10 and the cancer contrast enhancement particles 20 flow in the blood vessel. The blood vessel contrast enhancement particles 10 have a particle size larger than the vascular endothelial cell gap Ga at the time of the occurrence of the EPR effect, and hence keep flowing in the blood vessel. The cancer contrast enhancement particles 20 have a particle size smaller than the gap Ga, and hence are accumulated in the cancer tissue through a stromal cell system upon passing through the gaps Ga. The ultrasonic diagnostic apparatus scans an imaging region including a cancer tissue of an object with ultrasonic waves when the user operates the ultrasonic probe or the ultrasonic diagnostic apparatus main body, generates an ultrasonic image, in real time, which indicates the spatial distribution of acoustic impedance differences in the imaging region, and displays the ultrasonic image on a display device in real time. In an early stage of injection of the blood vessel contrast enhancement particles 10 and cancer contrast enhancement particles 20, it is possible to observe the flows of the blood vessel contrast enhancement particles 10 and cancer contrast enhancement particles 20 on an ultrasonic image. That is, it is possible to observe how the cancer contrast enhancement particles 20 are perfused to the cancer tissue on the ultrasonic image. Note that since the transmission frequency of an ultrasonic wave in the stage in FIG. 14 is set to be lower than the frequency ft of a crushing frequency because the purpose of transmission of the ultrasonic wave is to ultrasonically scan the imaging region instead of crushing the cancer contrast enhancement particle 20.

As shown in FIG. 15, after the blood vessel contrast enhancement particles 10 and the cancer contrast enhancement particles 20 are injected, the blood vessel in an imaging region is filled with the blood vessel contrast enhancement particles 10, and the cancer tissue is filled with the cancer contrast enhancement particles 20. In this case, the luminance values of the ultrasonic image are in a saturated state, and hence it is difficult to observe the flows of the blood vessel contrast enhancement particles 10 and cancer contrast enhancement particles 20 on the ultrasonic image. The user issues an instruction to transmit crushing ultrasonic waves via the input device or the like of the ultrasonic diagnostic apparatus for the purpose of observing the reperfusion of the cancer contrast enhancement particles 20 to the cancer tissue. Upon receiving the instruction to transmit crushing ultrasonic waves, the ultrasonic diagnostic apparatus transmits crushing ultrasonic waves having the frequency ft to the imaging region from the ultrasonic probe. Upon transmitting the crushing ultrasonic waves, the ultrasonic diagnostic apparatus scans the imaging region with ultrasonic waves having a frequency lower than the frequency ft to generate an ultrasonic image in real time, and displays the ultrasonic image on the display device in real time. The cancer contrast enhancement particles 20 existing in the imaging region are crushed upon reception of crushing ultrasonic waves and disappear from the blood vessel. In contrast to this, the blood vessel contrast enhancement particles 10 existing in the imaging region are not crushed even upon receiving crushing ultrasonic wave, and hence keep flowing in the blood vessel. Consequently, the cancer contrast enhancement particles 20 are not depicted on an ultrasonic image immediately after the transmission of crushing ultrasonic waves, and only the blood vessel contrast enhancement particles 10 are depicted. Note that in the above description, crushing ultrasonic waves are transmitted from the ultrasonic diagnostic apparatus in response to the reception of a transmission start instruction from the user. However, the ultrasonic diagnostic apparatus may automatically transmit crushing ultrasonic waves at a predetermined timing.

As shown in FIG. 16, when crushing ultrasonic waves are transmitted, the cancer contrast enhancement particles 20 begin to be accumulated in the cancer tissue via the stromal system upon passing through vascular endothelial cell gaps. That is, the cancer contrast enhancement particles 20 begin to reperfuse to the cancer tissue. After the transmission of crushing ultrasonic waves, therefore, the blood vessel contrast enhancement particles 10 (i.e., the vascular system) are depicted on an ultrasonic image with high luminance, and the reperfusion of the cancer contrast enhancement particles 20 to the cancer tissue is depicted.

As has been described above, the contrast agent according to the modification contains the blood vessel contrast enhancement particles 10 for enhancing the contrast of a blood vessel of an object and the diseased tissue contrast enhancement particles (cancer contrast enhancement particles when a contrast enhancement target is a cancer tissue) 20 for enhancing the contrast of a diseased tissue such as a cancer tissue of the object. The blood vessel contrast enhancement particle 10 and the diseased tissue contrast enhancement particle 20 are liposomes containing gases. The blood vessel contrast enhancement particle 10 has a particle size larger than the vascular endothelial cell gap Ga at the time of the occurrence of the EPR effect, and a structural strength that makes the particle crushable by the crushing frequency fb. The diseased tissue contrast enhancement particle 20 has a particle size smaller than the gap Ga, and a structural strength that makes the particle crushable by the crushing frequency fs different from the crushing frequency fb.

With the above arrangement, it is possible to selectively crush the blood vessel contrast enhancement particle 10 and the diseased tissue contrast enhancement particle 20 by adjusting crushing frequencies for the blood vessel contrast enhancement particle 10 and the diseased tissue contrast enhancement particle 20. It is therefore possible to selectively observe the reperfusion of the blood vessel contrast enhancement particles 10 to the contrast enhancement target blood vessel and the reperfusion of the diseased tissue contrast enhancement particles 20 to the contrast enhancement target diseased tissue by using the ultrasonic diagnostic apparatus.

As has been described above, according to this embodiment, it is possible to provide a contrast agent which individually targets the vascular system and stromal system of a diseased tissue.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A contrast agent comprising a blood vessel contrast enhancement particle configured to enhance contrast of a blood vessel of an object and a diseased tissue contrast enhancement particle configured to enhance contrast of a diseased tissue of the object, the blood vessel contrast enhancement particle having a first particle size larger than a gap of vascular endothelial cells under an EPR effect, and the diseased tissue contrast enhancement particle having a second particle size smaller than the gap.

2. The agent of claim 1, wherein the blood vessel contrast enhancement particle is chemically modified to be bonded to albumin in the blood vessel.

3. The agent of claim 1, wherein the diseased tissue contrast enhancement particle includes a ligand configured to be specifically bonded to the diseased tissue.

4. The agent of claim 3, wherein the diseased tissue is a cancer cell, and
the ligand is configured to be specifically bonded to a protein existing on a surface of the cancer cell or inside the cancer cell.

5. The agent of claim 1, wherein the first particle size is larger than a standard value of a gap between vascular endothelial cells contained in an imaging target blood vessel in which an EPR effect is recognized.

6. The agent of claim 1, wherein the second particle size is smaller than a standard value of a gap between vascular endothelial cells contained in an imaging target blood vessel in which an EPR effect is recognized.

7. The agent of claim 1, wherein the first particle size is not less than 200 nm.

8. The agent of claim 1, wherein the second particle size is not more than 100 nm.

9. The agent of claim 1, wherein at least one of the blood vessel contrast enhancement particle and the diseased tissue contrast enhancement particle is chemically modified with polyethylene glycol.

10. The agent of claim 1, wherein the blood vessel contrast enhancement particle and the diseased tissue contrast enhancement particle have a contrast enhancement effect corresponding to an imaging principle of an imaging mechanism used to image the object.

11. The agent of claim 10, wherein the blood vessel contrast enhancement particle and the diseased tissue contrast enhancement particle are bonded to a material having a contrast enhancement effect corresponding to an imaging principle of an imaging mechanism used to image the object or contain a material having the contrast enhancement effect.

12. The agent of claim 11, wherein the material having the contrast enhancement effect contains a material selected from the group consisting of iodine, gadolinium, gold, and bismuth in the case of that the imaging mechanism is a photon counting CT apparatus.

13. The agent of claim 11, wherein the material having the contrast enhancement effect includes a radionuclide in the case of that the imaging mechanism is one of a PET apparatus and a SPECT apparatus.

14. The agent of claim 11, wherein the material having the contrast enhancement effect contains one of a paramagnetic metal and a superparamagnetic iron oxide particle in the case of that the imaging mechanism is a magnetic resonance imaging apparatus and performs imaging using a difference between a longitudinal relaxation time and a transverse relaxation time.

15. The agent of claim 11, wherein the material having the contrast enhancement effect contains a paramagnetic metal belonging to lanthanoid elements in the case of that the imaging mechanism is a magnetic resonance imaging apparatus and performs imaging using a CEST effect.

16. The agent of claim 11, wherein the material having the contrast enhancement effect contains iodide in the case of that the imaging mechanism is one of an X-ray computed tomographic apparatus and an X-ray diagnostic apparatus.

17. The agent of claim 10, wherein the blood vessel contrast enhancement particle and the diseased tissue contrast enhancement particle comprise a lipid bilayer membrane containing a gas in the case of that the imaging mechanism is an ultrasonic diagnostic apparatus.

18. The agent of claim 1, wherein the blood vessel contrast enhancement particle and the diseased tissue contrast enhancement particle contain materials having contrast enhancement effects different from each other.

19. The agent of claim 18, wherein a first material having a contrast enhancement effect which is contained in the blood vessel contrast enhancement particle and a second material having a contrast enhancement effect which is contained in the diseased tissue contrast enhancement particle have contrast enhancement effects different from each other in an imaging principle of the same imaging mechanism.

20. The agent of claim 18, wherein a material having a first contrast enhancement effect which is contained in the blood vessel contrast enhancement particle exhibits a contrast enhancement effect in an imaging principle of a first imaging mechanism, and a material having a second contrast enhancement effect which is contained in the diseased tissue contrast enhancement particle exhibits a contrast enhancement effect in a second imaging mechanism using an imaging principle different from that of the first imaging mechanism.

21. The agent of claim 20, wherein when the first imaging mechanism is an X-ray computed tomographic apparatus and the second imaging mechanism is a photon counting CT apparatus, the material having the first contrast enhancement effect comprises iodine, and the material having the second contrast enhancement effect comprises a radionuclide.

22. The agent of claim 20, wherein when the first imaging mechanism is an X-ray computed tomographic apparatus and the second imaging mechanism is a PET apparatus, the material having the first contrast enhancement effect comprises iodine, and the material having the second contrast enhancement effect comprises a radionuclide.

23. The agent of claim 20, wherein when the first imaging mechanism is a magnetic resonance imaging apparatus and the second imaging mechanism is a photon counting CT apparatus, the material having the first contrast enhancement effect comprises one of a paramagnetic metal and a superparamagnetic iron oxide particle, and the material having the second contrast enhancement effect comprises a radionuclide.

24. The agent of claim 1, wherein the blood vessel contrast enhancement particle and the diseased tissue contrast enhancement particle comprise a lipid bilayer membrane containing a gas, the blood vessel contrast enhancement particle has a frequency which makes the particle crushable by a first frequency, and the diseased tissue contrast enhancement particle has a frequency which makes the particle crushable by a second frequency different from the first frequency.

25. The agent of claim 1, wherein at least one of the blood vessel contrast enhancement particle and the diseased tissue contrast enhancement particle comprises a lipid bilayer membrane which contains a material having a contrast enhancement effect or to which a material having a contrast enhancement effect is bonded.

26. The agent of claim 1, wherein at least one of the blood vessel contrast enhancement particle and the diseased tissue contrast enhancement particle comprises a colloidal particle made of a plurality of block copolymers each including a hydrophobic segment and a hydrophilic segment, and a material having a contrast enhancement effect is bonded to one of the hydrophobic segment and the hydrophilic segment.

27. The agent of claim 1, wherein at least one of the blood vessel contrast enhancement particle and the diseased tissue contrast enhancement particle comprises a dendrimer made of a plurality of unit molecular structures extending from a center and bonded to each other in a tree form, and a material having a contrast enhancement effect is bonded to a unit molecular structure, of the plurality of unit molecular structures, which is located at a terminal side.

28. The agent of claim 1, wherein the blood vessel contrast enhancement particles and the diseased tissue contrast enhancement particles are mixed such that an abundance of the blood vessel contrast enhancement particles is larger than an abundance of the diseased tissue contrast enhancement particles in the entire contrast agent, when the blood vessel contrast enhancement particles and the diseased tissue contrast enhancement particles are used to detect a treatment target.

29. The agent of claim 1, wherein the blood vessel contrast enhancement particles and the diseased tissue contrast enhancement particles are mixed such that an abundance of the blood vessel contrast enhancement particles is smaller than an abundance of the diseased tissue contrast enhancement particles in the entire contrast agent, when the blood vessel contrast enhancement particles and the diseased tissue contrast enhancement particles are used to determine a treatment effect.
